(19) United States
(12) Patent Application Publication
(10) Pub. No.: US 2003/0181877 A1
(43) Pub. Date: Sep. 25, 2003

(54) Container for needleless injection of medically active substances

(76) Inventors: Jochen Heinz, Vendersheim (DE); Michael Spallek, Ingelheim (DE)

Correspondence Address: Jacox Meckstroth & Jenkins, 2310 Far Hills Building, Dayton, OH, 45419-1575, US

(21) Appl. No.: 10/221,052

(22) PCT Filed: Mar. 8, 2001
(86) PCT No.: PCT/DE01/00875

(30) Foreign Application Priority Data
Mar. 11, 2000 (DE) 100 11 987.5

(51) Int. Cl. A61M 5/32

(52) U.S. Cl. 604/272

(57) ABSTRACT

Such receptacles are integrated or may be integrated with pressure means and at the exit-side end (2) comprise a closable exit opening (3) for the liquid medical substance filled in the receptacle. With known receptacles the exit opening (3) is difficult to close in a germ-tight and fluid tight manner. Furthermore a relatively high pressure must be exerted on the medically effective substance in order to penetrate all skin layers of the patient. For avoiding these disadvantages the invention envisages integrally forming a hollow-spike-like continuation (5) of a predetermined length corresponding to the thickness of the dermis of the human being at the exit side end (2), said continuation comprising the exit opening (3) and extending the exit channel. A conventional closure cap may be placed in a simple manner on this continuation (5) in a fluid-tight and germ-tight manner. Since the dermis is also penetrated with the receptacle with the hollow-spike-like continuation, a low pressure is sufficient in order for the ejected medical substance to penetrate the remaining skin layers.

### BACKGROUND OF THE INVENTION

The invention relates to a receptacle for the needle-less injection of medically effective substances which is integrated or may be integrated with a pressure means and which at the end on the exit side comprises a closable exit opening for the respective substance.

For the subcutaneous injection of medicaments, including diagnostic fluids, typically one uses syringes with an injection needle by way of which, when injecting, the skin of the patient is pierced. Since there are numerous patients who have a severe dislike to injection needles, so-called needle phobia, and because the injection needles are typically coated with silicone oil and therefore when injecting, silicone oil may get inside the body of the patient, one has developed devices for the needle-less injection of medically effective substances by way of which the substance to be injected without a needle may be “shot” through the skin of the patient by way of high pressure and deposited in the tissue which lies beneath this.

Devices for the needle-less injection of medicaments are known. Thus WO 98/499 94 as well as U.S. Pat. No. 5,704,911 and WO 97/278 34 describe corresponding devices with integrated or integratable receptacles which are suitable as a storage receptacle for medicaments in which by way of accompanying means such as a piston under spring bias, one may muster the required injection pressure in the case of application. These receptacles have an exit opening for the medically effective substance with a planar, flat edge which must be closed in a fluid-tight and germ-tight manner during storage.

Such receptacles which are manufactured of glass or plastic however all have considerable disadvantages. Thus the closure on the front side at the exit opening is very difficult to be designed in a fluid-tight and germ-tight manner. Furthermore when injecting, a slipping of the exit opening easily occurs on account of the planar contact resting and thus there occur undefined injection locations which medically is not particularly desirable. In the extreme case, on account of the slipping of the receptacle during the injection even incised injuries may occur.

A further disadvantage lies in the fact that in the receptacle one must build up a very high pressure since on injection all layers of skin must be penetrated by the substance in particular the outer skin layer, the dermis with the highest resistance.

### SUMMARY OF THE INVENTION

It is the object of the invention to further develop the previously mentioned receptacle such that its exit opening may be sealed with syringe-typical and conventional closure means and that a precise local injection with little pressure is possible.

Proceeding from the initially described receptacle for the needle-less injection of medically effective substances which is integrated or may be integrated with a pressure means and which at the end on the exit side comprises a closable exit opening for the respective substance, according to the invention this object is achieved in that on the end on the exit side there is integrally formed a hollow-spike-like continuation of a predetermined length corresponding to the thickness of the dermis of the human being, said continuation comprising the exit opening and extending the exit channel.

The invention exploits the fact that the dermis which opposes the needle-less injection with the highest resistance is not circulated with blood and contains no body fluid or nerves. It may therefore be penetrated by the hollow-spike-like continuation of the exit opening of the receptacle when placing the receptacle onto the skin without the patient experiencing anything unpleasant or body fluids exiting.

The advantages which may be achieved by the invention are:

1. The sealing of the receptacle may be effected with simple elastomer components by pushing the elastomer components onto the hollow-spike-like continuation, similar to ready manufactured syringes. These ready manufactured syringe closures are for example shown in the cap of R. D. Anand “Pharmazeutische Industrie” (“Pharmaceutical industry”) 54 No. 1, 1992, “Spritzampullen: Ein Vergleich in Zusammensetzung, Verarbeitung und Gebrauch vorgefüllter Spritzen” (“Injection ampullae: A comparison in composition, processing and use of prefilled syringes”), page 69-page 73, and serve the closure of ready manufactured syringes with and without an integrated needle.

2. On injection the receptacle may be positioned on the skin in a slip-secure manner by way of the hollow-spike-like continuation capable of piercing, since it may be placed onto the skin without injury; the contact point may be clearly felt.

3. The dermis is penetrated by the hollow-spike in a mechanostatic manner so that the injection jet may penetrate into deeper skin layers without being retarded.

4. Surprisingly it has been ascertained that a back-injection of tissue fluid is largely prevented.

5. The forces necessary for mustering the required forces, e.g., spring forces, for the drive of the movable piston may be reduced. Thus a more simple design of the application device is possible.

6. By way of the mechanostatic penetration of the dermis which is greatly dependent on the age and type of the patient, the injection method may be applied universally and leads to a great extent to reproducible results which may be compared over the most varied of patients.

According to a first embodiment of the invention the hollow-spike-like continuation is formed cylindrically, which in particular permits a favorable flow distribution of the fluid to be applied.

Particular advantages are achieved when the base of the hollow-spike-like continuation on the side of the receptacle is formed conically. This formation permits a slip-free positioning of the receptacle during application.

In order to ensure a simple penetration of the dermis, the hollow-spike-like continuation is preferably sharpened at its exit end. At the same time the hollow-spike-like continuation may either taper acutely in a symmetrical manner or also alternatively in an asymmetrical manner.

In order for the medically effective substance to remain in the hollow-spike-like continuation without a noticeable compromise of the pressure, according to a further development of the invention the receptacle is designed such that the exit channel in the hollow-spike-like continuation over its whole length from the exit opening on the end of the receptacle at the exit side up to the exit-side end of the continuation has a constant diameter.

The flow in the hollow-spike-like continuation may be influenced in a favorable manner when a flow body is incorporated in the exit channel congruently to its longitudinal axis.

Preferably the wall thickness of the unpointed hollow-spike-like continuation is circumferentially constant, which amongst other things permits a simple manufacture.

The receptacle however alternatively, with an asymmetrical tip, may be designed such that the wall thickness of the unsharpened hollow-spike-like continuation in the region of the projecting tip is greater that in the remaining region. With such a formation the danger of a breaking off of the projecting tip is less in the case of application.

The invention is hereinafter described in more detail by way of embodiment examples shown in the drawings.

### DESCRIPTION OF THE ACCOMPANYING DRAWINGS

There are shown in:

FIG. 1 in an enlarged longitudinal section representation the exit-side end of the receptacle according to the invention, with a hollow-spike-like continuation which symmetrically tapers in a pointed manner with an outer inclination,

FIG. 2 a representation corresponding to FIG. 1 with a hollow spike-like continuation which tapers asymmetrically in a pointed manner,

FIG. 3 a representation corresponding to FIG. 1 with a hollow spike-like continuation, which tapers symmetrically in a pointed manner with a funnel-like inner inclination,

FIG. 4 a representation according to FIG. 2 with which the wall thickness of the hollow-spike-like continuation in the region of the projecting asymmetrical tip is greater than in the remaining region,

FIG. 5 a representation according to FIG. 1 with a flow body in the exit channel of the hollow-spike-like continuation, and

FIG. 6 a representation according to FIG. 2, but without the conical, receptacle-side base of the hollow-spike-like continuation.

### DETAILED DESCRIPTION OF THE INVENTION

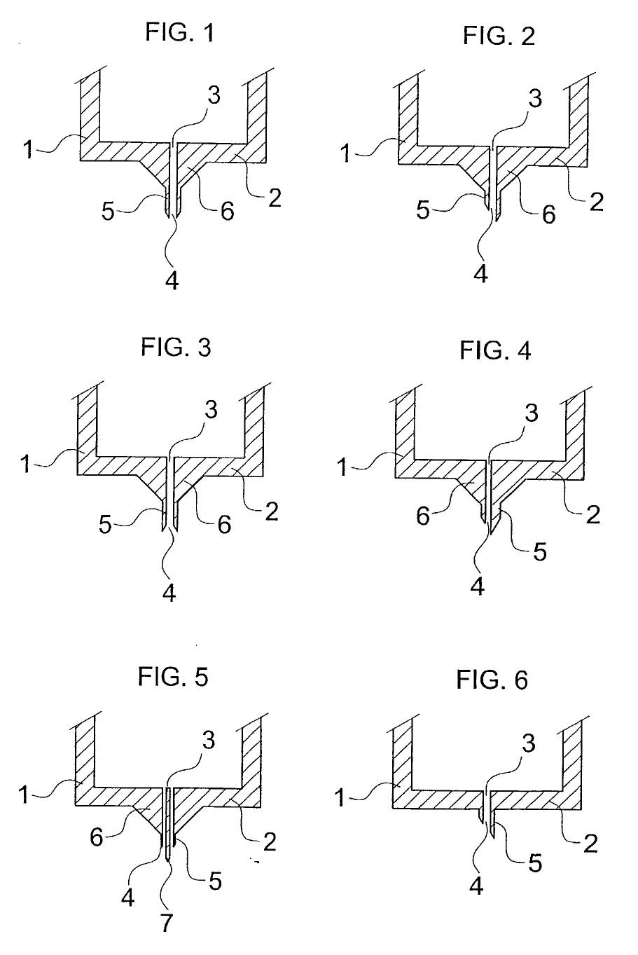

Since with the receptacle according to the invention for the needle-less injection of medically effective substances which is integrated or may be integrated with a pressure means and which at the end on the exit side comprises a closable exit opening for the respective substance it is only a question of the design of the exit opening, in the FIGS. 1-6 there are only shown various alternatives for shaping this exit opening. The remaining part of the receptacle, including the necessary pressure means, e.g. a piston under a high spring tension which may be incorporated into the cylindrical receptacle, is formed conventionally, for example corresponding to the initially mentioned state of the art. This part of the container therefore does not need to be described in more detail here.

In the figures there are shown in each case the wall of the exit-side end of the receptacle which is preferably formed cylindrically. This receptacle with all variants has an exit-side end 2 with an exit opening 3 formed therein for a medical substance with which the receptacle is filled.

At the same time the receptacle may be manufactured of glass or plastic, preferably of pharma-compatible plastic such as COC.

At the end 2 on the exit side a hollow-spike-like, cylindrical continuation 5 is integrally formed, which tapers in a pointed manner, comprises the exit opening 3 and extends the exit channel 4. In the FIGS. 1, 3 and 5 the continuation at the same time tapers in a pointed manner symmetrically and in the case of FIGS. 2, 4 and 6 asymmetrically.

The diameter of the exit channel 4 is constant over the whole length of the continuation 5 and in particular at its exit end has no widening of cross section since this could compromise the pressure conditions in an undesirable manner.

With the embodiment forms according to the FIGS. 1-5 at the same time the hollow-spike-like continuation 5 has a conical base 6 which serves for the slip-secure positioning of the receptacle at the location which is to be injected.

With the embodiment according to FIG. 1 the hollow-spike-like, cylindrical continuation 5 at the exit side is pointed towards the longitudinal axis, whilst with the embodiment form according to FIG. 2 the exit end is pointed in a funnel-shaped manner. The medically effective difference between the embodiment forms according to the FIGS. 1 and 2 lies in the fact that the active ingredient to be injected is deposited differently in the tissue and thus offers different pharmacological possibilities to the pharmacist.

With the embodiment form according to FIG. 5 with which the exit end analogously to FIG. 1 is pointed towards the longitudinal axis, a flow body 7 is incorporated in the exit channel 4 which effects a homogenization of the exiting medically effective substance with the application procedure. Furthermore one achieves the dispensing of the medicament over a larger area.

The embodiment forms according to the FIGS. 2, 4 and 6 with the asymmetrically pointedly tapering hollow-spike-like continuations 5 differ in particular in the shaping of the tip which with the embodiment according to FIG. 2 and FIG. 6 has the shape of an obliquely sectioned cylinder with a circumferentially equal wall thickness and in the case of FIG. 4 likewise has the shape of an obliquely sectioned cylinder, but with a wall thickness which is greater on half the side.

The medically effective differences between the embodiment forms according to the FIGS. 2, 4 and 6 on the one hand and in comparison to the embodiment forms according to the FIGS. 1, 3 and 5 on the other hand are the already mentioned possibilities of achieving various types of application when injecting.

The length of the hollow-spike-like continuation 5 typically corresponds to the thickness of the skin layer which is to be penetrates at the desired injection location. It is dimensioned such that the continuation merely penetrates the dermis, wherein by way of differently long continuations one may take account of the different skin types of the individual patients.

As is evident from the figures, the receptacle with the medically effective substance may be sealed fluid-tightly and germ-tightly in a simple and effective manner by way of simply placing a conventional protective cap onto the hollow-spike-like continuation, in particular of an electro metric continuation, as is used for protecting the needle of conventional ready manufactured syringes.

While the form of apparatus herein described constitutes a preferred embodiment of this invention, it is to be understood that the invention is not limited to this precise form of apparatus, and that changes may be made therein without departing from the scope of the invention which is defined in the appended claims.

1. A receptacle for the needle-less injection of medically effective substances which is integrated or may be integrated with a pressure means and which at the end (2) on the exit side comprises a closable exit opening (3) for the respective substance, characterized in that on the end (2) on the exit side there is integrally formed a hollow-spike-like continuation (5) of a predetermined length corresponding to the thickness of the dermis of the human being, said continuation comprising the exit opening (3) and extending the exit channel (4).

2. A receptacle according to claim 1, characterized in that the hollow-spike-like continuation (5) is formed cylindrically.

3. A receptacle according to claim 1 or 2, characterized in that the receptacle-side base (6) of the hollow-spike-like continuation (5) is formed conically.

4. A receptacle according to one of the claims 1 to 3, characterized in that the hollow-spike-like continuation tapers pointedly symmetrically towards its exit end.

5. A receptacle according to one of the claims 1 to 3, characterized in that the hollow-spike-like continuation (5) tapers pointedly asymmetrically towards its exit end.

6. A receptacle according to one of the claims 1 to 5, characterized in that the exit channel (4) in the hollow-spike-like continuation (5) over its whole length from the exit opening (3) at the exit-side end of the receptacle up to the exit-side end of the continuation (5) has a constant diameter.

7. A receptacle according to one of the claims 4 or 6, characterized in that a flow body (7) is incorporated in the exit channel (4) congruently to its longitudinal axis.

8. A receptacle according to claim 4 or to one of the claims 5 to 7, characterized in that the wall thickness of the unpointed hollow-spike-like continuation (5) is circumferentially constant.

9. A receptacle according to claim 5 and claim 6, characterized in that the wall thickness of the unpointed hollow-spike-like continuation (5) in the region of the projecting tip is greater than in the remaining region.
